(19) United States
(12) Patent Application Publication
(10) Pub. No.: US 2007/0106148 A1
(43) Pub. Date: May 10, 2007

(54) Electronic circuits to improve the sensitivity of magnetic resonance tracking catheters and intraluminal RF coils

(76) Inventor: Charles Lucian Dumoulin, Ballston Lake, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/261,990

(22) Filed: Oct. 28, 2005

(51) Int. Cl. A61B 5/05

(52) U.S. Cl. 600/410

(57) ABSTRACT

Devices, methods, and articles of manufacture related to an invasive device for inserting an inductor coil proximate to a specimen for detecting magnetic resonance imaging (MRI) resonant signals from the specimen; a conductor for conducting a signal from the inductor coil wherein the conductor has an effective electrical length set to be an odd multiple of λ/4 where λ is the wavelength of a known frequency of an electromagnetic signal presented to the conductor.

### FIELD OF THE INVENTION

This invention relates to the field of magnetic resonance imaging (MRI) and in particular, to an invasive interventional device such a guidewire, wire, or catheter for use with a magnetic resonance imaging (MRI) scanner.

### BACKGROUND OF THE INVENTION

Many conventional medical procedures involve imaging of internal organs of a subject during a medical procedure in order to guide a physician around structures such as vessels, and nerves.

There are also invasive medical procedures in which a device, such as a catheter has a radiofrequency (RF) coil incorporated in it for the purpose of acquiring magnetic resonance (MR) signals to be used either for locating the catheter and/or providing an MR image.

Patients undergoing medical imaging must be protected from failures of the medical imaging apparatus and other medical equipment connected to the patient. Due to the nature of magnetic resonance (MR) imaging, special requirements exist. In non-interventional (no devices are inserted into the subject) MR imaging, isolation of the patient from the MR imaging system is accomplished by incorporating insulating materials in the construction of surfaces which the patient is likely to touch (e.g. surface coils, patient bed, etc.).

For interventional procedures, the issue of patient isolation is more complicated since interventional devices are in contact with the subject. This can be especially critical if the invasive device is in contact with electrically-sensitive tissue (e.g. cardiac muscle; brain tissue, etc.).

In an MRI process, a sample, such as a human body, is placed in a magnetic field (the Bₒ field) that remains substantially constant throughout the MRI process. The magnetic moment of nuclei in the body, in particular nuclei of hydrogen, become aligned with the magnetic field. Next the sample is exposed to an oscillating magnetic field having a selected frequency in the radio frequency (rf) region of the electromagnetic spectrum, causing the nuclei in the sample to resonate. The rf radiation is then switched off, but the nuclei continue to resonate resulting in the emission of rf radiation from the resonating nuclei. The emission is detected as an MRI signal.

The resonance frequency of the sample depends upon the strength of the large magnetic field. This frequency is called the Larmor frequency and is expressed by the relationship L=γH, where L is the Larmor frequency, H is the strength of the magnetic field, and γ is a constant dependent upon the particular nuclei. The oscillating rf field is generated by a transmit rf coil that typically encloses the sample. The frequency of the applied oscillating rf field is chosen to be substantially the same as the Larmor frequency.

The transmit rf coil may also be used to receive the resonating emissions from the sample. Alternatively, a separate and distinct receive coil such as a surface coil may be used to receive the resonating emissions. The inductance and capacitance of the transmit and receive rf coils determine the tuned frequency of each coil and the impedance of each coil. The coil impedance of the transmit coil is matched to the optimum source impedance of the transmit amplifier to minimize the power required to resonate the nuclei. The coil impedance of the receive coil is matched to the optimum source impedance of the MRI system preamplifier so that the noise figure of the MRI system receive chain is minimized. The receive rf coil has its maximum sensitivity for the detecting emitted rf radiation when the inductance and capacitance of the rf coil are chosen so that the rf coil has a tuned frequency which is the same as the Larmor frequency of emitted rf radiation.

Prior art receive coils have, under certain conditions, had problems tuning the frequency of the receive rf coil to the Larmor frequency of the nuclei. Since the coil includes reactive elements (coil inductance and capacitive elements) the impedance of the coil is frequency dependent. Coil impedance has real and imaginary components that vary with frequency. Most coils have their impedance tuned and matched to maximize the signal-to-noise ratio of the detected signal. Often it is sufficient to adjust tuning and matching capacitors upon construction of the rf coil to match the source impedance of the preamplifier which minimizes the preamplifier's noise figure.

For a receive rf coil with a fixed geometry, the signal-to-noise ratio of magnetic resonance signals from a sample increases approximately linearly with the MRI scanner's magnetic field, B₀. The closer the receive rf coil is to the sample, the larger the signal and the signal-to-noise ratio. Thus, for low fields it is very important that the receive coil be close to the body. In general, the greater the distance between the coil and body, the poorer the MRI image.

Another factor affecting resonance frequency is variability in the value of the main magnetic field. Superconducting magnets produce high, stable magnetic fields (typically greater than 0.3T). Permanent magnets or conventional electromagnets can produce fields which often vary with time due to temperature variations, resulting in changes in the resonance frequency. Drifts in the magnetic field for a permanent magnet can be of the order of a thousand parts-per-million (PPM) per degree Centigrade resulting in a shift in the Larmor frequency. Such drifts result in the coil being severely detuned with respect to the Larmor frequency of the MRI system, leading to poor signal-to-noise ratios and, hence, poor image quality.

There are a number of techniques for tuning transmit rf and receive rf coils to the resonance frequency of the MRI system. See, for example, U.S. Pat. No. 4,897,604 which shows an expandable rf coil composed of a two parts including a main section and a removable bridge segment. Different size bridge segments change both the active and the physical circumference of the coil. In U.S. Pat. No. 5,143,068 a flexible coil having a fixed physical size is tuned by an externally located coupling coil circuit that has variable capacitors. U.S. Pat. No. 4,791,372 also relies upon variable capacitors.

One aspect of magnetic resonance tracking methods used in invasive medical procedures is that the interventional device incorporates a cable or wire to bring magnetic resonance signals detected within the body out to the imaging and tracking system. An MR imaging or tracking coil is placed within or on the surface of a subject and is typically connected to an external receiver by a coaxial cable. One consequence of placing conducting material within the body during a magnetic resonance procedure is that the radiofrequency (RF) pulses generated by the transmit rf coil and used for image formation and device tracking, can induce currents in the conductor. These currents can result in the creation of strong electric fields at the ends of the conductor. If the end of the conductor is surrounded by conducting tissue such as blood, these strong electric fields will induce currents in the tissue and cause heating. The amount of heating within the tissue is related to the power and duty cycle of the RF pulse. Stronger and more frequently applied RF pulses create greater amounts of heating. The amount of heating is also indirectly related to the strength of the static magnetic field used in magnetic resonance procedures since greater RF power is required to nutate the nuclear spins in higher magnetic fields.

If the amount of heat deposited within conducting tissue near an interventional device causes a temperature rise of less than about four degrees Celsius, then no damage to the tissue will occur. If the heat deposited causes the temperature to rise in excess of about four degrees Celsius, however, reversible or irreversible tissue damage can occur.

It should be noted that placement of wires or other conducting structures within the body during a magnetic resonance examination may be desirable for reasons other than tracking of a device. For example, small magnetic resonance receive coils can be used to make images of localized anatomy such as the wall of a blood vessel. Alternatively, it may be desirable to place other medical devices such as endoscopes or catheter guide-wires which could result in localized tissue heating during magnetic resonance exams.

In many invasive medical procedures it is desirable to incorporate miniature receive rf coils in the invasive device. While it may be desirable to include reactive elements such as capacitors in the construction of such devices to maximize the signal-to-noise ratio of the magnetic resonance signals detected by the invasive device, the need to minimize the dimensions of the invasive device frequently makes the incorporation of reactive elements difficult or impossible. Consequently, there is a need for invasive device designs that allow for reactive elements such as capacitors without adversely affecting device size.

### BRIEF DESCRIPTION OF THE INVENTION

An embodiment may comprise a self-disabling probe device comprising: a probe; an inductor coil located in the probe for detecting magnetic resonance imaging (MRI) resonant signals; a cable connected to the inductor coil wherein the cable has a length set to be an odd-multiple of λ/4 where λ is the wavelength of a known RF high power pulse signal used during an MRI procedure; and at least one diode in electrical contact with the cable wherein the diode is structured to conduct during the RF high power pulse signal to a ground, thereby self-disabling the coil during RF high power pulses by raising impedance in the cable.

An embodiment may also comprise a method for improving the sensitivity of and for reducing the adverse heating effects of an invasive probe having an inductor coil used in detecting magnetic resonance imaging (MRI) resonant signals comprising: providing a cable connected to an inductor coil wherein the cable has an effective electrical length set to be an odd multiple of λ/4 where λ is the wavelength of a known RF high power pulse signal used during an MRI procedure; and providing at least one diode in electrical contact with the cable wherein the diode conducts to a ground when the RF high power pulse signal is present thereby self-disabling the coil during RF high power pulses by raising the impedance in the cable thereby reducing adverse heating effects, and thereby improving the sensitivity of the coil by having the diode not conduct during the detection via the coil of lower power MRI resonant signals.

An embodiment may also comprise a self-disabling device comprising: an invasive device for inserting an inductor coil proximate to a specimen for detecting magnetic resonance imaging (MRI) resonant signals from the specimen; a conductor for conducting a signal from the inductor coil wherein the conductor has an effective electrical length set to be an odd multiple of λ/4 where λ is the wavelength of a known frequency of an electromagnetic signal presented to the conductor; and at least one electrical device in electrical contact with the conductor wherein the electrical device is structured to conduct to ground when the known frequency of an electromagnetic signal is presented to the conductor thereby self-disabling the inductor coil when the known frequency of an electromagnetic signal is presented by raising the impedance in the conductor.

An embodiment may also comprise an article of manufacture for use with Magnetic Resonance Imaging (MRI) comprising: a length of a conductor connectable to an MRI sensor wherein the conductor has an effective electrical length set to be an odd multiple of λ/4 where λ is the wavelength of a known frequency of an electromagnetic signal presented to the conductor during an MRI procedure.

### BRIEF DESCRIPTION OF THE DRAWINGS

The following figures should not be construed to limiting of the invention in any way. Like elements are numbered alike in the several Figures.

FIG. 1 is a perspective, partially cut-away view of an embodiment for tracking the location of a device in a subject.

FIG. 2 is a schematic illustration showing an RF coil incorporated into a device for insertion into the body of a subject in a fashion well known to those skilled in the art.

FIGS. 3-8 are schematic diagrams of exemplary circuit embodiments.

FIG. 9 shows an embodiment of a prior art catheter having an RF coil therein following the embodiment shown in FIG. 2.

FIG. 10 shows a close-up of the catheter shown in FIG. 9.

FIG. 11 shows a prior art circuit and coil.

FIG. 12 shows a prior art circuit and coil.

### DETAILED DESCRIPTION OF THE INVENTION

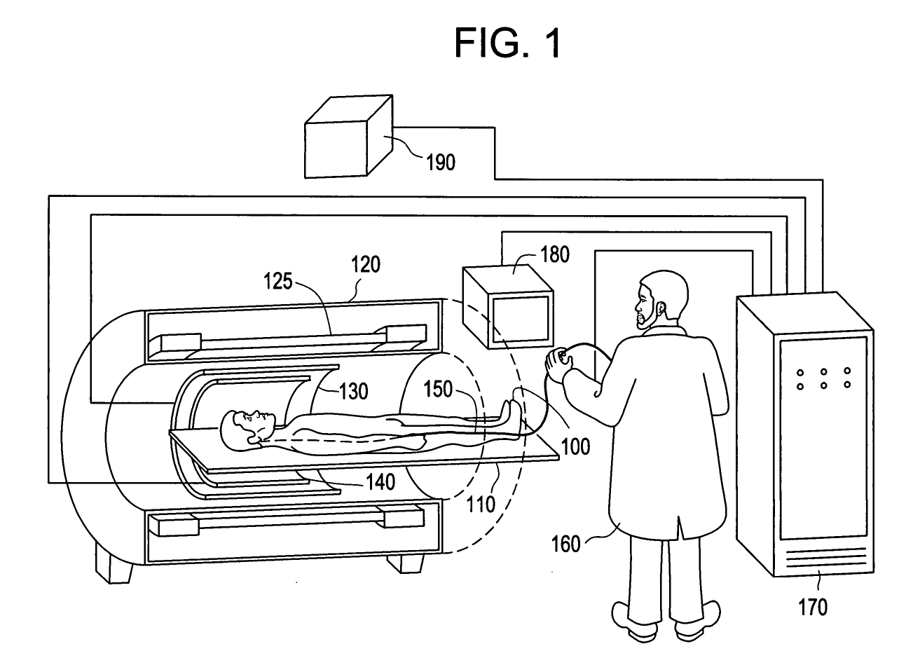

In FIG. 1, a subject 100 on a support table 110 is placed in a homogeneous magnetic field generated by a magnet 125 in magnet housing 120. Magnet 125 and magnet housing 120 have cylindrical symmetry and are shown sectioned in half to reveal the position of subject 100. A region of subject 100 into which a device 150, shown as a catheter, is inserted, is located in the approximate center of the bore of magnet 125. Subject 100 is surrounded by a set of cylindrical magnetic field gradient coils 130 (shown sectioned in half) which create magnetic field gradients of predetermined strength at predetermined times. Gradient coils 130 generate magnetic field gradients in three mutually orthogonal directions.

An external coil 140 also surrounds a region of interest of subject 100. Coil 140 is shown (sectioned in half) as a cylindrical external coil which has a diameter sufficient to encompass the entire subject. Other geometries, such as smaller cylinders specifically designed for imaging the head or an extremity can be used instead. Non-cylindrical external coils, such as surface coils, may alternatively be used. External coil 140 radiates radiofrequency energy into subject 100 at predetermined times and at a predetermined frequency so as to nutate nuclear magnetic spins of atomic nuclei of subject 100 in a fashion well known to those skilled in the art. The nutation of the spins causes them to resonate at the Larmor frequency. The Larmor frequency for each spin is directly proportional to the strength of the magnetic field experienced by the spin. This field strength is the sum of the static magnetic field generated by magnet 125 and the local field generated by magnetic field gradient coil 130.

Device 150 is inserted into subject 100 by an operator 160, and may be a guide wire, a catheter, an endoscope, a laparoscope, a biopsy needle or other device. This device contains an RF or inductor coil 10 which detects MR signals generated in the subject responsive to the radiofrequency field created by external coil 140. Since the RF or inductor coil 10 is small, the region of sensitivity is also small. Consequently, the detected signals have Larmor frequencies which arise only from the strength of the magnetic field in the immediate vicinity of the coil. These detected signals are sent to an imaging and tracking unit 170 where they are analyzed. The position of device 150 is determined in imaging and tracking unit 170 and is displayed on a display means 180. In the preferred embodiment of the invention the position of device 150 is displayed on display means 180 by superposition of a graphic symbol on a conventional MR image driven by a superposition means within imaging and tracking unit 170. In alternative embodiments of the invention, the graphic symbol representing device 150 is superimposed on diagnostic images obtained from an imaging means 190 which may be an X-ray, a computed tomography (CT), a Positron Emission Tomography or ultrasound imaging device. Other embodiments of the invention display the position of the device numerically or as a graphic symbol without reference to a diagnostic image.

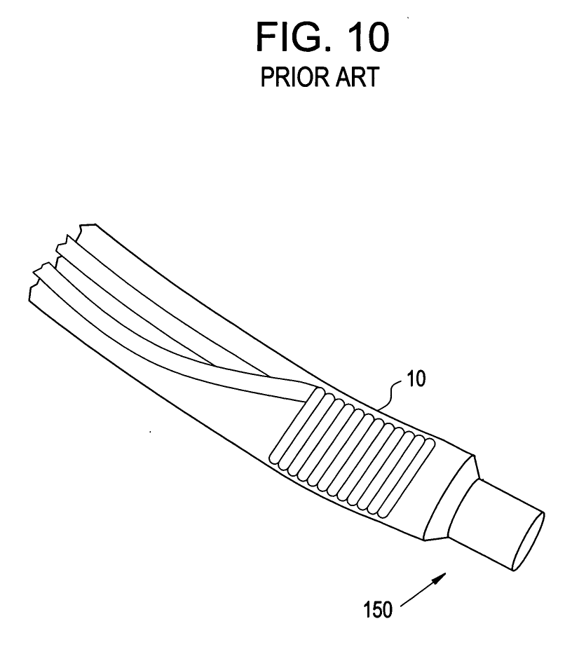

FIG. 11 is a schematic diagram of simple prior art MR tracking catheter circuit. The circuit shown in FIG. 11 has several important features. First, the coil 10 is untuned, but operates at a frequency below its self-resonance. Second, no attention is paid to the length of the coaxial cable 25. Typically, this cable is simply made as long as the device.

In active MR device tracking and luminal imaging a small pickup coil 10 is used to acquire MR signals from inside the body. To date, most devices that have been constructed have a simple circuit comprising a pickup coil, co-axial cable and a connector as shown in FIG. 11. While these devices are simple to construct and relatively insensitive to changes in the local magnetic environment (e.g. a metallic guidewire can be placed in the coil without a large drop in signal), they do not provide the optimum sensitivity to MR signals.

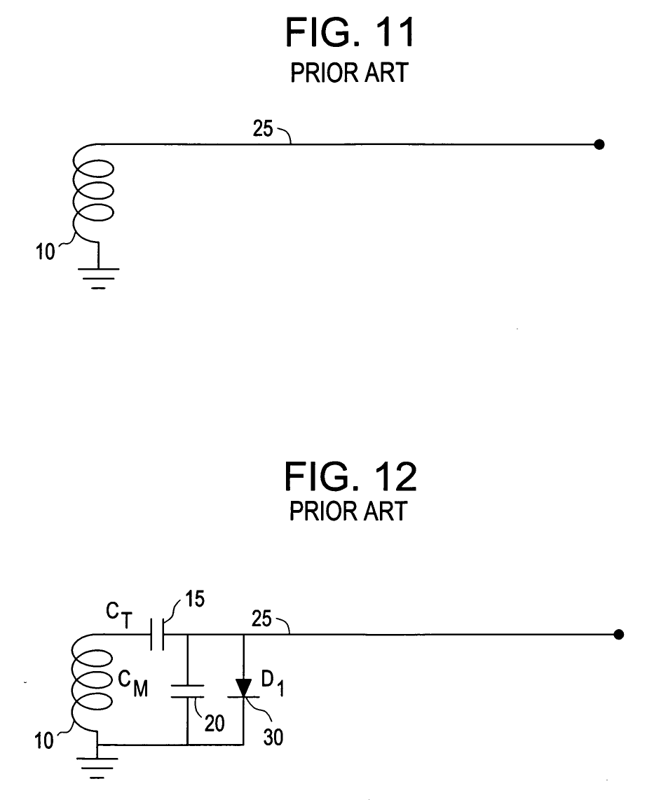

In response, tuned resonance circuits and other variations of have been proposed to address several issues. These proposals follow the examples found in the state of the art for receive and transmit rf coils. For example, resonant coils and dynamic disabling circuits have been built in which a DC bias is placed on the device to detune the coil. This strategy comes from the conventional designs for surface coils in MR imaging. An example of such an approach is shown in prior art FIG. 12.

In FIG. 12, a tuning capacitor, C_{T}, and a matching capacitor, C_{M}, are added to the circuit to resonate the coil 10. Since the coil 10 is a resonant coil system, it will couple strongly to the excitation RF coil of the MR scanner (typically the body coil) during excitation. This will cause the excitation magnetic field in the vicinity of the coil 10 to be much greater than desired. It can also lead to undesired heating of the coil 10. Consequently, a decoupling diode, D₁, (which is sometimes resonated with its own inductor in other versions of the circuit) is added to detune the coil. The decoupling diode D₁ detunes the coil when an appropriate DC bias is applied to the diode D₁ from an appropriate control circuit.

While this active DC bias current control approach works adequately for surface coils, there are several disadvantages when it is used in catheter coils. For example, the addition of two capacitors and a diode can significantly add to the size, bulk and complexity of the device. Also, the safety ramifications of having a DC bias current present within the device during insertion into the patient must be considered.

Variations in the size and tissue composition of the anatomy placed in an imaging coil affect the amount of RF energy getting into and the amount of signal detected from the imaged anatomy. For these reasons the RF coil should be tuned whenever it is known that the composition of the anatomy or material in the coil changes. Tuning the probe or catheter entails adjusting two types of capacitors on the RF probe. One capacitor is called the matching capacitor and the other the tuning capacitor. The matching capacitor matches the impedance of the coil with imaged object to that of the cable coming from the measurement device such as a spectrometer. The tuning capacitor changes the resonance frequency of the RF coil.

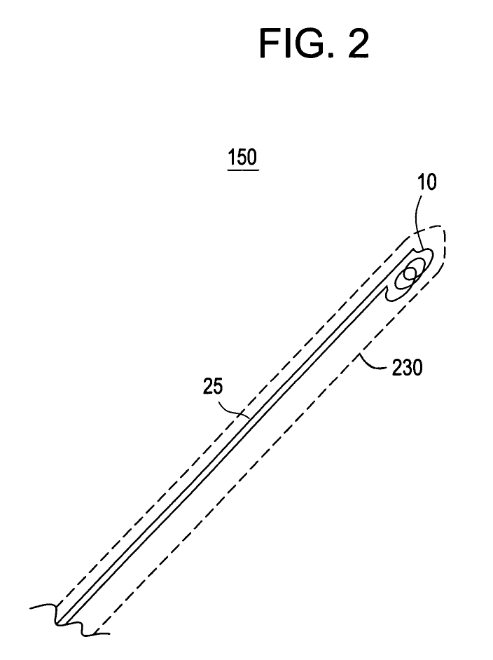

For understanding, one example of a possible embodiment of device 150 is shown in greater detail in FIG. 2. A small RF coil 10 is electrically coupled to the MR system via cable 25. The cable which may be a coaxial cable for example, is encased in an outer shell 230 of device 150. The MR signal arising from the tissue surrounding device 150 is detected by coil 10. Coil 10 in this embodiment is a receive only coil. Using the catheter device 150 a technique for guiding interventional procedures with MR imaging can be achieved. For example, active real-time position monitoring of catheters with MR imaging is made possible by incorporating a small RF coil into the tip of the catheter. This can be used instead of fluoroscopy methods for example.

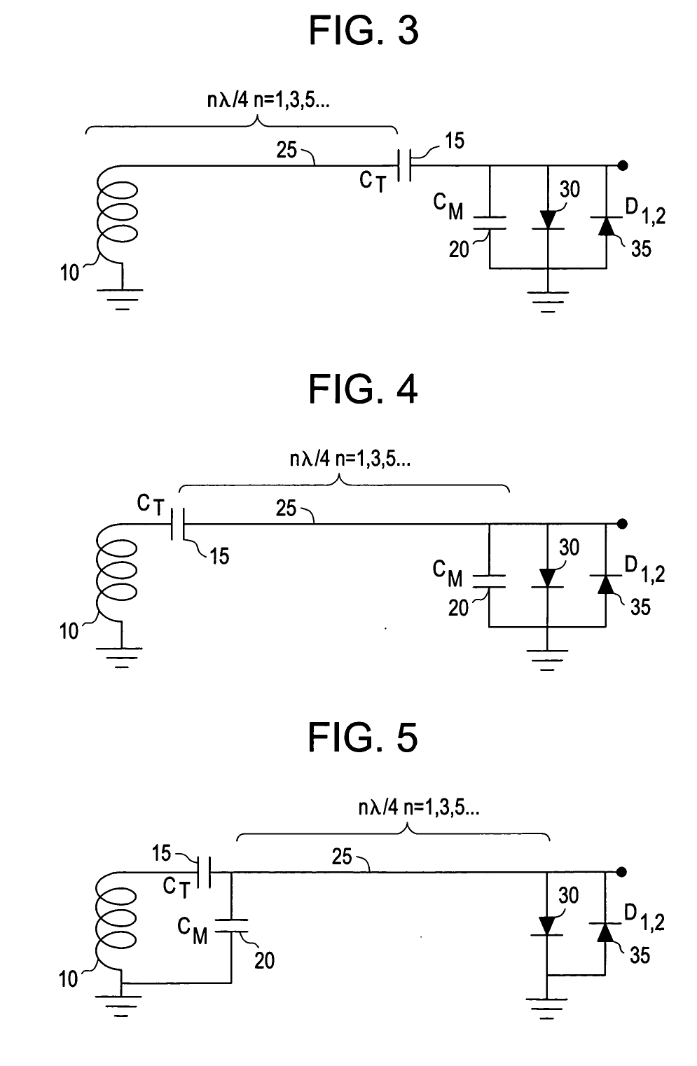

As shown in FIGS. 3-8, “self-disabling” exemplary circuits overcome the limitations of requiring an externally applied DC bias control in the device by incorporating a tuned length of coaxial cable 25 and crossed diodes (30, 35). The cable 25 is chosen to be an odd-integral multiple of λ/4 where λ is the wavelength of the electromagnetic signal in the coaxial cable. This length is chosen to exploit the property of transmission lines and coaxial cables in which the impedance is inverted every λ/4 along the cable. This property can be understood by considering the destructive interference of a reflected signal on the cable. For example, consider a sinusoidal rf signal that is inserted into one end of a coaxial cable that is λ/4 long and shorted to ground at the opposite end. The short at the opposite end will cause the rf signal to be reflected. Since the cable is λ/4 long, the reflected signal will have traveled a distance of λ/2 (i.e. λ/4+λ/4) by the time it returns to the injection point, and thus will be 180 degrees out of phase with the injected signal. This phase cancellation will make the cable appear to be open-circuited (i.e. have high impedance) at the injection point, even though (and indeed because of) a short (or low impedance) exists at the other end of the cable.

The crossed diodes (30, 35) at the end of a λ/4 cable provide a dynamic impedance. When the diodes (30, 35) conduct (as they will during a “high power” RF pulse), the impedance of the diodes (30, 35) becomes low and the impedance at the end of the λ/4 cable 25 becomes high (thereby self-disabling the coil). Conversely, when the diodes (30, 35) do not conduct (as they will during quiescent periods and during the reception of MR signals), the impedance of the diodes (30, 35) is high and the impedance at the end of the λ/4 cable 25 becomes low (thereby self-enabling the coil).

Some of the embodiments of the present invention also incorporate tuning and matching capacitors located at the coil 10 or located at the connector end of the device. FIGS. 3-8 show several different embodiments of circuits having different locations of the tuning capacitor 15, the matching capacitor 20 and diodes 30. However the invention is not limited to these embodiments. Additionally, both series and parallel resonance circuits are contemplated depending upon the application. Resonance occurs when the reactance of an inductor balances the reactance of a capacitor at some given frequency. In such a resonant circuit where it is in series resonance, the current will be maximum and offering minimum impedance. In parallel resonant circuits the opposite occurs

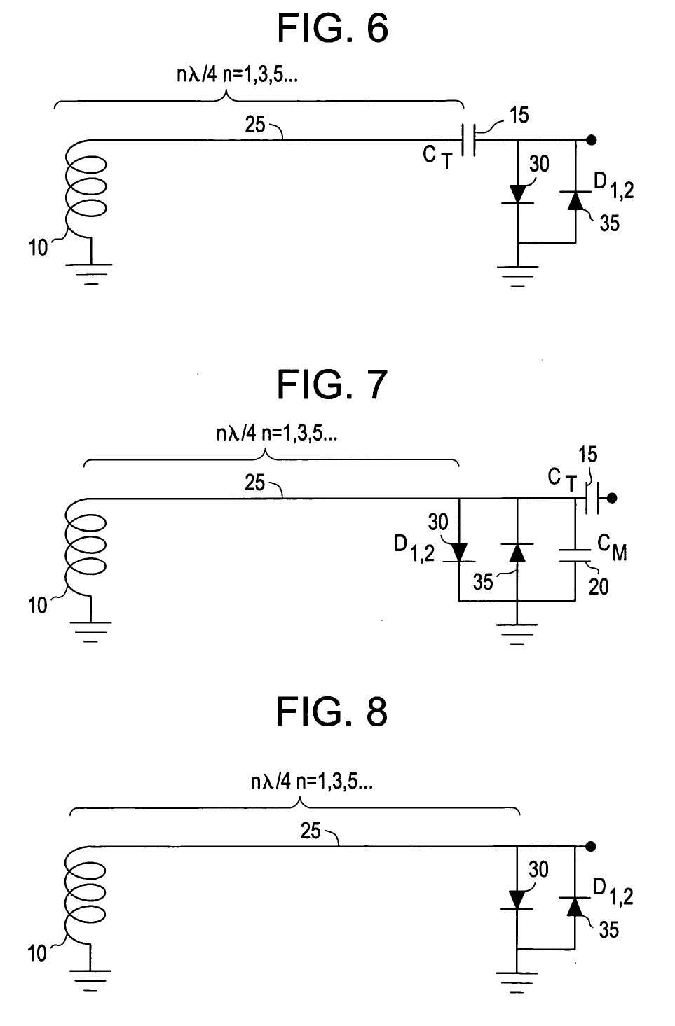

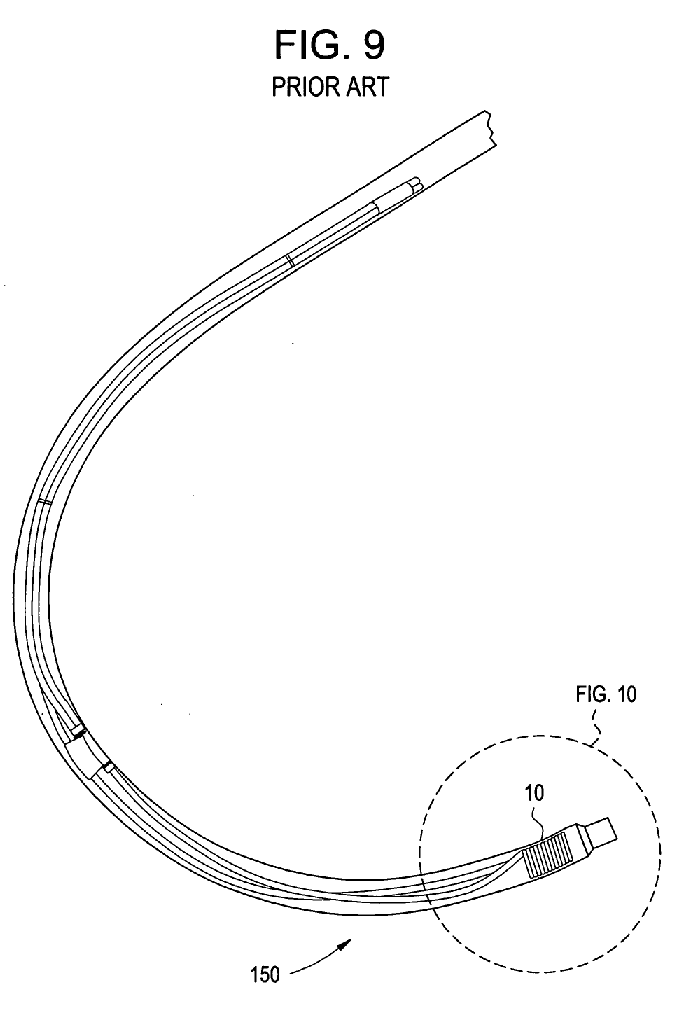

The sensitivity of the coil 10 is also improved by the circuits shown in FIGS. 3-8 in comparison to the prior art. The coil 10 needs to have a frequency close to the Larmor frequency. However, because the coil 10 is very small in a catheter application for example, it is a challenge to tune the Larmor frequency remotely. Locating the resonance circuit capacitors 20 and diodes 30 remotely from the coil 10 to tune the resonance circuit remotely is shown in FIGS. 3, 6, 7, and 8, for example and decreases the size of the catheter to be inserted into the patient. Examples of prior art catheters shown to scale having a small coil 10 located in their distal end are shown in FIGS. 9 and 10. With the current invention (particularly the embodiment shown in FIG. 3), these coils could be tuned (and hence offer greater signal-to-noise ratio) without increasing the size of the catheter.

Therefore, the circuits shown in FIGS. 3-8 make use of the principle that at λ/4 the impedances become inverted. Thus, cable 25 is a made of an odd multiple of λ/4 length and when it is shorted at one end, the other end will behave as an open (disabled circuit). Additionally, by providing crossed diodes (30, 35), the diodes will effectively conduct the signal at a high power stage when the RF excitation pulse is applied. This disables the coil 10 because the impedance at the end of the cable becomes high when the crossed diodes conduct. The arrangement also reduces the overall heating effect as well because the coil 10 is disabled during high power RF pulses. This also increases sensitivity by allowing the coil 10 to only operate as receive only coil at the lower power stages when lower power resonance frequencies propagating from the sample in the patient are to be detected with the coil 10 using the resonant and tuned circuit enabled by the capacitors, i.e., when the high power RF pulses are not occurring.

Several circuits for improveed sensitivity MR tracking catheters and intraluminal RF coils have been shown in FIGS. 3-8. The circuits obtain high sensitivity by resonating the inductance of the pickup coil 10 with a capacitor, i.e., a tuned resonant circuit. To minimize device size, however, the capacitor(s) (15, 20) may be located remotely from the inductor coil 10. This creates a low profile catheter. Diodes 30 are also added to the circuit to disable the coil 10 during high-power events such as RF pulses. The combination of a tuned coil 10 and self-disabling permits high sensitivity without the introduction of a DC bias control voltage which would pose a safety hazard to the patient. This combination also produces an increase signal to noise ratio.

The present invention will become an important part of the construction of active MR tracking devices. The designs described here will be useful at least for MR guided catheters, guidewires and endoscopes. However, the approach is not limited to coils placed in devices. It can also be used with dipole antenna guidewires and if desired, with conventional surface coils.

Note that some of the variations of this “self-disabling” invention could be used in conjunction with an active decoupling scheme (such as those used in conventional surface coils) if desired by simply removing a single diode 35. For example, those configurations in which the tuning capacitor, C_{T}, 15 is placed between the diodes 30, 35 and the λ/4 cable might be useful without the risk of exposing the patient to the DC bias from the control circuitry since the tuning capacitor, C_{T}, will serve as a DC block. Since C_{T} is placed in the proximal end of the device (i.e. in the connector end as shown in FIGS. 3 and 6), the device can be constructed in a fail-safe fashion.

Another important aspect of an embodiment of the disclosed invention is the use of cables having effective electrical lengths that are multiples of λ/4. It is well know to those skilled in the art that lump element circuits composed of discrete inductors and capacitors can be used to phase shift an RF signal by any desired amount, and thus simulate a cable of arbitrary or desired physical length. Thus, a segment of Nλ/4 cable can be constructed using a shorter length of cable combined with a lumped element circuit to provide a circuit whose effective electrical length or electrical properties are identical to a Nλ/4 cable and yet is physically shorter than a Nλ/4 cable. It is the intention of the inventors to include the use of such lumped element circuits within the scope of embodiments of the present invention.

This written description uses examples to disclose the invention, including the best mode, and also to enable any person skilled in the art to make and use the invention. The patentable scope of the invention is defined by the claims, and may include other examples of devices, methods, and articles of manufacture that occur to those skilled in the art. Such other examples are intended at least to be within the scope of the claims if they have structural elements that do not differ from the literal language of the claims, or if they include equivalent structural elements with insubstantial differences from the literal languages of the claims and/or as allowed by law.

Furthermore, the skilled artisan will recognize the interchangeability of various features from different embodiments. Similarly, the various features described, as well as other known equivalents for each feature, can be mixed and matched by one of ordinary skill in this art.

What is claimed is:

1. A self-disabling probe device comprising:
a probe;
an inductor coil located in the probe for detecting magnetic resonance imaging (MRI) resonant signals;
a cable connected to the inductor coil wherein the cable has a length set to be an odd-multiple of λ/4 where λ is the wavelength of a known RF high power pulse signal used during an MRI procedure; and
at least one diode in electrical contact with the cable wherein the diode is structured to conduct during the RF high power pulse signal to a ground, thereby self-disabling the coil during RF high power pulses by raising impedance in the cable.

2. The device of claim 1 further comprising:
at least one capacitor to tune the inductor coil and to create a resonant circuit.

3. The device of claim 1 wherein the at least one diode is located remotely from the coil.

4. The device of claim 2 wherein the at least one capacitor is located remotely from the coil.

5. The device of claim 1 wherein the probe comprises a catheter.

6. The device of claim 1 wherein the probe comprises a guidewire.

7. A method for improving the sensitivity of and for reducing the adverse heating effects of an invasive probe having an inductor coil used in detecting magnetic resonance imaging (MRI) resonant signals comprising:
providing a cable connected to an inductor coil wherein the cable has an effective electrical length set to be an odd multiple of λ/4 where λ is the wavelength of a known RF high power pulse signal used during an MRI procedure; and
providing at least one diode in electrical contact with the cable wherein the diode conducts to a ground when the RF high power pulse signal is present thereby self-disabling the coil during RF high power pulses by raising the impedance in the cable thereby reducing adverse heating effects, and thereby improving the sensitivity of the coil by having the diode not conduct during the detection via the coil of lower power MRI resonant signals.

8. A self-disabling device comprising:
an invasive device for inserting an inductor coil proximate to a specimen for detecting magnetic resonance imaging (MRI) resonant signals from the specimen;
a conductor for conducting a signal from the inductor coil wherein the conductor has an effective electrical length set to be an odd multiple of λ/4 where λ is the wavelength of a known frequency of an electromagnetic signal presented to the conductor; and
at least one electrical device in electrical contact with the conductor wherein the electrical device is structured to conduct to ground when the known frequency of an electromagnetic signal is presented to the conductor thereby self-disabling the inductor coil when the known frequency of an electromagnetic signal is presented by raising the impedance in the conductor.

9. The device of claim 8 further comprising:
at least one capacitor to tune the inductor coil and to create a resonant circuit.

10. The device of claim 8 wherein the at least one electrical device is a diode.

11. The device of claim 9 wherein the at least one capacitor is located remotely from the inductor coil.

12. The device claim 8 wherein the invasive device comprises a catheter.

13. The device of claim 8 wherein the invasive device comprises a guidewire apparatus.

14. The device of claim 8 wherein the at least one electrical device comprises a pair of diodes.

15. An article of manufacture for use with Magnetic Resonance Imaging (MRI) comprising:
a length of a conductor connectable to an MRI sensor wherein the conductor has an effective electrical length set to be an odd multiple of λ/4 where λ is the wavelength of a known frequency of an electromagnetic signal presented to the conductor during an MRI procedure.

16. The self-disabling device of claim 8 where the conductor for conducting a signal from the inductor coil has an effective electrical length set to be an odd multiple of λ/4, where λ is the wavelength of a known frequency of an electromagnetic signal presented to the conductor, is further in electrical contact with at least one:
lump element circuit structured to electrically simulate the effective electrical length of the conductor thereby permitting different physical lengths of the conductor to be used while retaining the effective electrical length set to be an odd multiple of λ/4, where λ is the wavelength of a known frequency of an electromagnetic signal presented to the conductor.
